(19) United States
(12) Patent Application Publication
(10) Pub. No.: US 2013/0005658 A1
(43) Pub. Date: Jan. 3, 2013

(54) MICRO-RNA REGULATION IN ISCHEMIA AND ISCHEMIA-REPERFUSION INJURY

(75) Inventors: Eric N. Olson, Dallas, TX (US); Eva Van Rooij, Boulder, CO (US); Daniel Quiat, Dallas, TX (US)

(73) Assignee: Board of Regents, The University of Texas System, Austin, TX (US)

(21) Appl. No.: 13/516,189

(22) PCT Filed: Dec. 15, 2010
(86) PCT No.: PCT/US2010/060460

§ 371 Date: Sep. 5, 2012

Related U.S. Application Data
(60) Provisional application No. 61/286,622, filed on Dec. 15, 2009.

(51) Int. Cl. A61K 31/7088; A61P 9/10; A61K 38/17; A61P 7/10

(52) U.S. Cl. 514/16.4; 514/44 A

(57) ABSTRACT

The present invention relates to the identification of miRNAs that are involved in cardiac remodeling following ischemia and ischemia reperfusion injury. A subset of these miRNAs are regulated in the short term following an ischemic event indicating that these miRNAs play an important role in the induction of subsequent pathological events. Modulation of these identified miRNAs as a treatment or prevention for myocardial ischemia and ischemia reperfusion injury is described.

### CROSS-REFERENCE TO RELATED APPLICATIONS

This application claims the benefit of priority of U.S. Provisional Application No. 61/286,622, filed Dec. 15, 2009, which is herein incorporated by reference in its entirety.

### STATEMENT OF GOVERNMENT SUPPORT

This invention was made with government support under Grant Number HL53351-06 awarded by the National Institutes of Health. The government has certain rights in the invention.

### FIELD OF THE INVENTION

The present invention relates generally to the fields of cardiology, pathology and molecular biology. In particular, the invention encompasses several miRNAs that are regulated in response to ischemia and reperfusion of ischemic cardiac tissue. Manipulation of the expression of these identified miRNAs provides a novel therapeutic approach for treatment of myocardial ischemia and other forms of ischemic injury.

### BACKGROUND OF THE INVENTION

Heart disease and its manifestations, including coronary artery disease, myocardial infarction, congestive heart failure and cardiac hypertrophy, clearly present a major health risk in the United States today. The cost to diagnose, treat and support patients suffering from these diseases is well into the billions of dollars. A particularly severe manifestation of heart disease is myocardial infarction. Myocardial infarction (MI), more commonly known as a heart attack, is a medical condition that occurs when the blood supply to a part of the heart is interrupted, most commonly due to rupture of a vulnerable plaque. The interruption in blood supply initially results in ischemia and oxygen shortage causing damage, which can progress into death of heart tissue (i.e. infarct). It is the leading cause of death for both men and women throughout the world. In the United States alone, coronary heart disease is responsible for 1 in 5 deaths, and some 7,200,000 men and 6,000,000 women are living with some form of coronary heart disease. Of these, 1,200,000 people suffer a new or recurrent coronary attack every year, and about 40% of them die as a result of the attack. This means that roughly every 65 seconds, an American dies of a coronary event.

A precursor to myocardial infarction is myocardial ischemia. Myocardial ischemia occurs when oxygen delivery cannot meet myocardial metabolic requirements in the heart. This deficiency can result from either a reduced supply of oxygen (decreased coronary bloodflow) or an increased myocardial demand for oxygen (increased wall stress or afterload). Although hypoxia is an obligatory component, it is not the sole environmental stress experienced by the ischemic heart. Ischemia produces a variety of environmental stresses that impair cardiovascular function. As a result, multiple signaling pathways are activated in mammalian cells during ischemic injury in an attempt to minimize cellular injury and maintain cardiac output. Among the transcriptional regulators activated are members of the hypoxia inducible factor (HIF) transcription factor family. In response to decreased oxygen concentration, HIF factors regulate a variety of genes that affect a myriad of cellular processes including metabolism (enhanced glucose uptake), formation of new blood vessels via angiogenesis, cell survival, and oxygen delivery, all of which are important in the heart. These gene expression cascades are rapid and influence the initial response to myocardial ischemia, which impacts the resulting decrease in cardiac contractility. Ischemia is often followed by reperfusion of the tissue allowing the re-admission of oxygen and metabolic substrates which replace the ischemic metabolites. The process of reperfusion induces biochemical, structural and functional changes in the myocardium and may tip the balance between cell survival and cell death.

Changes in gene expression and signaling pathways associated with post-MI remodeling have been intensively studied, with the goal of identifying therapeutic targets that might allow restoration of function to the injured heart. Recently, key roles of microRNAs in cardiac hypertrophy and heart failure have been described, pointing to a new mode of regulation of cardiac disease (van Rooij et al. (2006) Proc Natl Acad Sci USA, Vol. 103(48):18255-60; van Rooij and Olson (2007) J Clin Invest., Vol. 117(9):2369-76; van Rooij et al. (2008) Proc Natl Acad Sci USA, Vol. 105(35):13027-32). MicroRNAs (miRNAs) are small, non-protein coding RNAs of about 18 to about 25 nucleotides in length that are derived from individual miRNA genes, from introns of protein coding genes, or from poly-cistronic transcripts that often encode multiple, closely related miRNAs. See review by Carrington et al. (Science, Vol. 301(5631):336-338, 2003). MiRNAs act as repressors of target mRNAs by promoting their degradation, when their sequences are perfectly complementary, or by inhibiting translation, when their sequences contain mismatches.

MiRNAs are transcribed by RNA polymerase II (pol II) or RNA polymerase III (pol III; see Qi et al. (2006) Cellular & Molecular Immunology, Vol. 3:411-419) and arise from initial transcripts, termed primary miRNA transcripts (pri-miRNAs), that are generally several thousand bases long. Pri-miRNAs are processed in the nucleus by the RNase Drosha into about 70- to about 100-nucleotide hairpin-shaped precursors (pre-miRNAs). Following transport to the cytoplasm, the hairpin pre-miRNA is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

Based on genetic studies in mice and humans, it is becoming increasingly clear that miRNAs are indeed actively involved in cardiac remodeling, growth, conductance, and contractility (reviewed in van Rooij and Olson (2007) Journal of Clinical Investigation, Vol. 117(9):2369-2376). Cardiac ischemia induces remodeling that can influence the function of the ventricle and the prognosis for survival, which is dependent on the degree of myocyte loss and the extent of remodeling of the surviving myocardial tissue. Identification and characterization of miRNAs involved in initial cellular remodeling processes in response to ischemia can provide novel therapeutic approaches to reduce or eliminate the maladaptive effects of an ischemic insult.

### SUMMARY OF THE INVENTION

The present invention is based, in part, on the discovery of several miRNAs that are regulated in cardiac tissue immediately following an ischemic event or ischemia followed by tissue reperfusion. Modulation of these identified miRNAs presents a novel therapeutic approach for treating myocardial ischemia and preventing the development of a myocardial infarction and heart failure. Accordingly, the invention provides a method of treating or preventing myocardial ischemia in a subject in need thereof comprising modulating the expression or activity of one or more of the identified miRNAs in the heart cells of the subject. In one embodiment, the one or more miRNAs are selected from the group consisting of a miR-15 family member, miR-21, miR-26a, let-7b, miR-199, miR-320, miR-214, miR-10a, miR-10b, miR-574, miR-92a, miR-499, miR-101a, miR-101b, miR-125b, miR-145, miR-126, a miR-30 family member, miR-143, miR-185, miR-34a, miR-1, miR-133, miR-210, and miR-29a-c. In certain embodiments, the subject has coronary artery disease.

In one embodiment, the method comprises administering to the subject an inhibitor of one or more of the identified miRNAs. For instance, the inhibitor can be an inhibitor of the expression or activity of a miRNA selected from the group consisting of a miR-15 family member, miR-92a, miR-320, miR-21, miR-199, miR-499, and a miR-30 family member. The inhibitor of one or more miRNAs can include an antagomir or an antisense oligonucleotide.

In another embodiment, the method comprises administering to the subject an agonist of one or more of the identified miRNAs. In some embodiments, the agonist increases the expression or activity of a miRNA selected from the group consisting of miR-126, miR-143, miR-210, and miR-29a-c. In certain embodiments, the agonist of one or more miRNAs is a polynucleotide comprising a mature sequence of the one or more miRNAs. The agonist can be expressed in vivo from an expression construct.

In some embodiments, the method of treating or preventing myocardial ischemia further comprises administering a second cardiac therapeutic agent. The second cardiac therapeutic agent can be an agent that is prescribed to treat angina or coronary artery disease. In one embodiment, the second cardiac therapeutic agent is selected from the group consisting of an antianginal agent, beta blocker, an ionotrope, a diuretic, ACE-I, AII antagonist, an endothelin receptor antagonist, an HDAC inhibitor, and a calcium channel blocker.

The present invention also includes a method of preventing or treating ischemia-reperfusion injury in a subject in need thereof. In certain embodiments, the method comprises administering a modulator of one or more miRNAs regulated following reperfusion injury. The modulator can be an inhibitor or agonist of miRNA function or expression.

In another embodiment, the present invention encompasses a method of preventing or reducing cardiomyocyte loss in response to hypoxia in a subject in need thereof comprising administering an inhibitor of miR-199, miR-320, and/or an agonist of miR-210 to the subject. In some embodiments, the agonist of miR-210 is the transcription factor, HIF1α.

### BRIEF DESCRIPTION OF THE DRAWINGS

FIG. 1. Expression of miR-199a in cardiac tissue following an ischemic insult. Real time PCR analysis for miR-199 of tissue isolated from the infarct zone of mice 6, 24, and 48 hours following induction of a myocardial infarction.

FIG. 2. HIF1α is upregulated in cardiomyocytes in response to miR-199 inhibition. A. Northern blot analysis of HIF1α expression in cardiomyocytes treated with an antimiR against miR-199a or a mismatch control (MM). B. Realtime PCR analysis for miR-199 in various tissues following intravenous injection of antimiR-199a or a mismatched control (MM) in mice.

FIG. 3. MiR-320 is downregulated in cardiac tissue following myocardial ischemia. Real time PCR analysis for miR-320 of tissue isolated from the infarct zone of mice 6, 24, and 48 hours following induction of myocardial ischemia.

FIG. 4. Expression of miR-210 is induced in cardiac cells following ischemia and hypoxia. A. Real time PCR analysis for miR-210 of tissue isolated from the infarct zone of mice 6, 24, and 48 hours following induction of myocardial ischemia. B. Real time PCR analysis for miR-210 of rat neonatal cardiomyocytes 6 and 12 hours following exposure to hypoxic conditions in vitro.

FIG. 5. Specific miRNAs are regulated in response to ischemia reperfusion. Heat map of statistically significant regulated miRNAs in ischemic tissue following reperfusion (p<0.01).

### DETAILED DESCRIPTION OF THE INVENTION

The present invention is based, in part, on the identification of a subset of miRNAs that are regulated immediately following an ischemic insult. In particular, the inventors found at least thirty miRNAs that were significantly upregulated in the ischemic tissue and at least thirty eight miRNAs that were downregulated in the ischemic tissue in the first forty eight hours following the ischemic event. A subset of the miRNAs exhibited a dynamic regulation immediately following the ischemic event: expression of some of the miRNAs was initially downregulated followed by an upregulation, while expression of others was initially upregulated followed by a downregulation. In addition, the inventors discovered an overlapping, but unique subset of miRNAs to be regulated in reperfused cardiac tissue following an ischemic event. At least thirty two miRNAs were found to be significantly upregulated, while at least forty eight miRNAs were significantly downregulated in the reperfused cardiac tissue. The overlap in regulated miRNAs suggests that miRNAs may be involved in different cardiac disease processes at different time points and can act to influence the disease state. Thus, there is a set of miRNAs that are involved in the response of the heart to ischemic injury and manipulation of the activity or expression of these specific miRNAs can result in the control of cardiac remodeling such that any potential resulting infarct is limited in size and cardiac contractility is maintained. Accordingly, the present invention provides a method of treating or preventing myocardial ischemia in a subject in need thereof comprising modulating the expression or activity of one or more miRNAs listed in Tables 1 and 2 in the heart cells of the subject. The invention also includes the corresponding human sequences of the miRNAs listed in Tables 1 and 2. In certain embodiments, the one or more miRNAs is selected from the group consisting of a miR-15 family member (e.g. miR-15a, miR-15b, miR-16-1, miR-16-2, miR-195, miR-424 and miR-497) (SEQ ID NOs: 1-6), miR-21 (SEQ ID NO: 7), miR-199a (SEQ ID NOs: 8-9), miR-320 (SEQ ID NO: 10), miR-214 (SEQ ID NO: 11), miR-10a (SEQ ID NO: 12), miR-10b (SEQ ID NO: 13), miR-574 (SEQ ID NOs: 14-15), miR-92a (SEQ ID NO: 16), miR-499 (SEQ ID NOs: 17-18), miR-101a/miR-101b (SEQ ID NO: 19), miR-126 (SEQ ID NO: 20), a miR-30 family member (e.g. miR-30a, miR-30b, miR-30c, miR-30d, and miR-30e) (SEQ ID NOs: 21-25), miR-143 (SEQ ID NO: 26), miR-185 (SEQ ID NO: 27), miR-34a (SEQ ID NO: 28), miR-1 (SEQ ID NO: 29), miR-133a/miR-133b (SEQ ID NOs: 30-31), miR-210 (SEQ ID NO: 32), miR-29a-c (SEQ ID NOs: 33-35), miR-26a (SEQ ID NO: 37), let-7b (SEQ ID NO: 38), miR-125b (SEQ ID NO: 39), and miR-145 (SEQ ID NO: 40).

As used herein, the term “modulate” refers to a change or an alteration in a biological activity of a miRNA. Modulation may be a change in the expression level of the miRNA, a change in binding characteristics of the miRNA (e.g. to a target mRNA or to a component of the RISC complex), or any other change in the biological or functional properties associated with the miRNA. Modulation can be either an increase or decrease in the expression or function of the miRNA. The term “modulator” refers to any molecule or compound which is capable of changing or altering the expression or biological activity of a miRNA as described above. A modulator can be an inhibitor of miRNA function or expression or it can be an agonist of miRNA function or expression.

As used herein, the term “myocardial ischemia” or “ischemia” refers to a condition within the heart that results from a deficient supply of blood to the myocardium. Ischemia can involve, for example, restricted blow flow to the heart tissue as a result of blockage or reduced flow through one or more coronary arteries that normally supplies the heart tissue. Ischemic damage includes loss of cardiomyocytes, cardiac hypertrophy, cardiomyopathy, reduction of cardiac contractility, and arrhythmia. An infarction results when the blood supply to a localized area is deprived for a prolonged period of time so that heart cells die. An “infarct” is an area of coagulation necrosis in a tissue resulting from obstruction of circulation to the area. Modulation of the expression or activity of one or more miRNAs disclosed herein in the heart tissue of a subject is effective at reducing or preventing ischemic damage, including preventing the development of an infarct or reducing infarct size, maintaining cardiac contractility, and minimizing cardiac remodeling in a subject that has experienced an ischemic event or that is at risk of experiencing an ischemic event.

Ischemia and the resulting ischemic damage in the heart are brought on by an ischemic event or injury. An “ischemic event” or “ischemic injury” is any instance that results, or could result, in a deficient supply of blood to the heart tissue. Ischemic events or injuries encompassed by the present invention include, but are not limited to, hypoglycemia, tachycardia, atherosclerosis, hypotension, thromboembolism, external compression of a blood vessel, embolism, Sickle cell disease, inflammatory processes, which frequently accompany thrombi in the lumen of inflamed vessels, hemorrhage, cardiac failure and cardiac arrest, shock, including septic shock and cardiogenic shock, hypertension, an angioma, and hypothermia.

In one embodiment, the method of treating or preventing myocardial ischemia in a subject in need thereof comprises administering an inhibitor of one or more miRNAs listed in Tables 1 and 2 to the subject. In another embodiment, the inhibitor is an inhibitor of the expression or activity of one or more miRNAs selected from the group consisting of a miR-15 family member (e.g. miR-15a, miR-15b, miR-16-1, miR-16-2, miR-195, miR-424 and miR-497) (SEQ ID NOs: 1-6), miR-92a (SEQ ID NO: 16), miR-21 (SEQ ID NO: 7), miR-199a (SEQ ID NOs: 8-9), miR-320 (SEQ ID NO: 10), miR-499 (SEQ ID NOs: 17-18), and a miR-30 family member (e.g. miR-30a, miR-30b, miR-30c, miR-30d, and miR-30e) (SEQ ID NOs: 21-25).

In certain embodiments, the inhibitor of one or more miRNAs is an antisense oligonucleotide. The antisense oligonucleotides can include ribonucleotides or deoxyribonucleotides or a combination thereof. Preferably, the antisense oligonucleotides have at least one chemical modification (e.g., sugar or backbone modification). For instance, suitable antisense oligonucleotides may be comprised of one or more “conformationally constrained” or bicyclic sugar nucleoside modifications (BSN) that confer enhanced thermal stability to complexes formed between the oligonucleotide containing BSN and their complementary microRNA target strand. For example, in one embodiment, the antisense oligonucleotides contain at least one “locked nucleic acid.” Locked nucleic acids (LNAs) contain the 2′-O, 4′-C-methylene ribonucleoside (structure A) wherein the ribose sugar moiety is in a “locked” conformation. In another embodiment, the antisense oligonucleotides contain at least one 2′, 4′-C-bridged 2′ deoxyribonucleoside (CDNA, structure B). See, e.g., U.S. Pat. No. 6,403,566 and Wang et al. (1999) Bioorganic and Medicinal Chemistry Letters, Vol. 9: 1147-1150, both of which are herein incorporated by reference in their entireties. In yet another embodiment, the antisense oligonucleotides contain at least one modified nucleoside having the structure shown in structure C. The antisense oligonucleotides targeting one or more miRNAs can contain combinations of BSN (LNA, CDNA and the like) or other modified nucleotides, and ribonucleotides or deoxyribonucleotides.

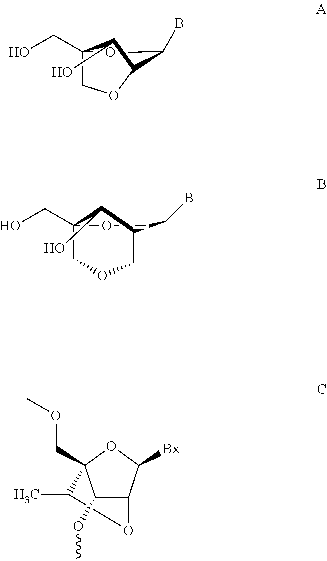

Alternatively, the antisense oligonucleotides can comprise peptide nucleic acids (PNAs), which contain a peptide-based backbone rather than a sugar-phosphate backbone. Other modified sugar or phosphodiester modifications to the antisense oligonucleotide are also contemplated. For instance, other chemical modifications that the antisense oligonucleotides can contain include, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). In one embodiment, antisense oligonucleotides targeting one or more miRNAs contain 2′O-methyl sugar modifications on each base and are linked by phosphorothioate linkages. Antisense oligonucleotides, particularly those of shorter lengths (e.g., less than 15 nucleotides) can comprise one or more affinity enhancing modifications, such as, but not limited to, LNAs, bicyclic nucleosides, phosphonoformates, 2′ O-alkyl modifications and the like. In some embodiments, suitable antisense oligonucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. For instance, to facilitate in vivo delivery and stability, the antisense oligonucleotide may be linked to a steroid, such as cholesterol moiety, a vitamin, a fatty acid, a carbohydrate or glycoside, a peptide, or other small molecule ligand at its 3′ end.

Preferable antisense oligonucleotides useful for inhibiting the activity of miRNAs are about 5 to about 25 nucleotides in length, about 10 to about 30 nucleotides in length, or about 20 to about 25 nucleotides in length. In certain embodiments, antisense oligonucleotides targeting one or more of the miRNAs described herein are about 8 to about 18 nucleotides in length, and in other embodiments about 12 to about 16 nucleotides in length. Any 8-mer or longer complementary to the target miRNA may be used, i.e., any antimiR complementary to the 5′ end of the miRNA and progressing across the full complementary sequence of the target miRNA. Antisense oligonucleotides can comprise a sequence that is at least partially complementary to a mature miRNA sequence from one or more miRNAs. “Partially complementary” refers to a sequence that is at least about 75%, 80%, 85%, 90%, 95%, 96%, 97%, 98%, or 99% complementary to a target miRNA sequence. In some embodiments, the antisense oligonucleotide can be substantially complementary to a mature miRNA sequence, that is at least about 90%, 95%, 96%, 97%, 98%, or 99% complementary to a target miRNA sequence. In one embodiment, the antisense oligonucleotide comprises a sequence that is 100% complementary to a mature miRNA sequence.

In some embodiments, the antisense oligonucleotides are antagomirs. “Antagomirs” are single-stranded, chemically-modified ribonucleotides that are at least partially complementary to at least one mature miRNA sequence. Antagomirs may comprise one or more modified nucleotides, such as 2′-O-methyl-sugar modifications. In some embodiments, antagomirs comprise only modified nucleotides. Antagomirs can also comprise one or more phosphorothioate linkages resulting in a partial or full phosphorothioate backbone. To facilitate in vivo delivery and stability, the antagomir can be linked to a cholesterol or other moiety at its 3′ end. Antagomirs suitable for inhibiting one or more miRNAs can be about 12 to about 70 nucleotides in length, about 15 to about 50 nucleotides in length, about 18 to about 35 nucleotides in length, about 19 to about 28 nucleotides in length, or about 20 to about 25 nucleotides in length. The antagomirs can be at least about 75%, 80%, 85%, 90%, 95%, 96%, 97%, 98%, or 99% complementary to a mature miRNA sequence. In some embodiments, the antagomir may be substantially complementary to a mature miRNA sequence, that is at least about 95%, 96%, 97%, 98%, or 99% complementary to a target polynucleotide sequence. In other embodiments, the antagomirs are 100% complementary to a mature miRNA sequence.

In another embodiment, the method of treating or preventing myocardial ischemia in a subject in need thereof comprises administering an agonist of one or more miRNAs listed in Tables 1 and 2 to the subject. In certain embodiments, the agonist is an agonist of one or more miRNAs selected from the group consisting of miR-126 (SEQ ID NO: 20), miR-143 (SEQ ID NO: 26), miR-210 (SEQ ID NO: 32), and miR-29a-c (SEQ ID NOs: 33-35).

As used herein, an “agonist” is a molecule or compound that enhances the expression or activity of a target miRNA. An agonist can be a polynucleotide encoding the miRNA sequence. For instance, in one embodiment, an agonist of one or more miRNAs is a polynucleotide comprising a mature sequence of the one or more miRNAs. In another embodiment, the agonist of one or more miRNAs can be a polynucleotide comprising the pri-miRNA or pre-miRNA sequence for the one or more miRNAs. The polynucleotide comprising the mature, pre-miRNA, or pri-miRNA sequence can be single stranded or double stranded. The polynucleotides may contain one or more chemical modifications, such as locked nucleic acids, peptide nucleic acids, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages. In some embodiments, the polynucleotide comprising one or more miRNA sequences is conjugated to a steroid, such as cholesterol, a vitamin, a fatty acid, a carbohydrate or glycoside, a peptide, or another small molecule ligand. In certain embodiments, an agonist of one or more miRNAs is an agent distinct from the miRNA itself that acts to increase, supplement, or replace the function of the one or more miRNAs.

The inhibitors and agonists of the miRNAs of the invention can be expressed in vivo from a vector. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, an expression vector for expressing an agonist of one or more miRNAs comprises a promoter “operably linked” to a polynucleotide encoding a sequence of the one or more miRNAs. The phrase “operably linked” or “under transcriptional control” as used herein means that the promoter is in the correct location and orientation in relation to a polynucleotide to control the initiation of transcription by RNA polymerase and expression of the polynucleotide. The polynucleotide encoding one or more miRNAs may encode the primary miRNA sequence, the precursor-miRNA sequence, the mature miRNA sequence, or the star (e.g. minor) sequence of the one or more miRNAs. The polynucleotide comprising a sequence of one or more miRNAs can be about 18 to about 2000 nucleotides in length, about 70 to about 200 nucleotides in length, about 20 to about 50 nucleotides in length, or about 18 to about 25 nucleotides in length.

Inhibitors of one or more miRNAs (e.g., antisense oligonucleotides) can be expressed from a vector in vivo. For instance, in one embodiment, an expression vector for expressing an inhibitor of one or more miRNAs comprises a promoter operably linked to a polynucleotide encoding an antisense oligonucleotide, wherein the sequence of the expressed antisense oligonucleotide is at least partially complementary to the mature sequence of one or more miRNAs.

As used herein, a “promoter” refers to a DNA sequence recognized by the synthetic machinery of the cell, or introduced synthetic machinery, required to initiate the specific transcription of a gene. Suitable promoters include, but are not limited to RNA pol I, pol II, pol III, and viral promoters (e.g. human cytomegalovirus (CMV) immediate early gene promoter, the SV40 early promoter, and the Rous sarcoma virus long terminal repeat). In one embodiment, the promoter is a tissue specific promoter. Of particular interest are muscle specific promoters, and more particularly, cardiac specific promoters. These include the myosin light chain-2 promoter (Franz et al. (1994) Cardioscience, Vol. 5(4):235-43; Kelly et al. (1995) J. Cell Biol., Vol. 129(2):383-396), the alpha actin promoter (Moss et al. (1996) Biol. Chem., Vol. 271(49): 31688-31694), the troponin 1 promoter (Bhaysar et al. (1996) Genomics, Vol. 35(1):11-23); the Na+/Ca2+ exchanger promoter (Barnes et al. (1997) J. Biol. Chem., Vol. 272(17):11510-11517), the dystrophin promoter (Kimura et al. (1997) Dev. Growth Differ., Vol. 39(3):257-265), the alpha7 integrin promoter (Ziober and Kramer (1996) J. Bio. Chem., Vol. 271(37):22915-22), the brain natriuretic peptide promoter (LaPointe et al. (1996) Hypertension, Vol. 27(3 Pt 2):715-22) and the alpha B-crystallin/small heat shock protein promoter (Gopal-Srivastava (1995) J. Mol. Cell. Biol., Vol. 15(12):7081-7090), alpha myosin heavy chain promoter (Yamauchi-Takihara et al. (1989) Proc. Natl. Acad. Sci. USA, Vol. 86(10):3504-3508) and the ANF promoter (LaPointe et al. (1988) J. Biol. Chem., Vol. 263(19):9075-9078).

In certain embodiments, the promoter operably linked to a polynucleotide encoding a miRNA family inhibitor may be an inducible promoter. Inducible promoters are known in the art and include, but are not limited to, tetracycline promoter, metallothionein IIA promoter, heat shock promoter, steroid/thyroid hormone/retinoic acid response elements, the adenovirus late promoter, and the inducible mouse mammary tumor virus LTR.

In some embodiments, the human cytomegalovirus (CMV) immediate early gene promoter, the SV40 early promoter, the Rous sarcoma virus long terminal repeat, rat insulin promoter, and glyceraldehyde-3-phosphate dehydrogenase promoter can be used to obtain high-level expression of the polynucleotide sequence of interest. The use of other viral or mammalian cellular or bacterial phage promoters, which are well-known in the art to achieve expression of a polynucleotide sequence of interest, is contemplated as well, provided that the levels of expression are sufficient for a given purpose.

Methods of delivering expression constructs and nucleic acids to cells are known in the art and can include, for example, calcium phosphate co-precipitation, electroporation, microinjection, DEAE-dextran, lipofection, transfection employing polyamine transfection reagents, cell sonication, gene bombardment using high velocity microprojectiles, and receptor-mediated transfection.

Preferably, administration of an inhibitor or agonist of one or more miRNAs listed in Tables 1 and 2 results in the improvement of one or more symptoms of myocardial ischemia, myocardial infarction, heart failure, or cardiac remodeling. The one or more improved symptoms can be, for example, reduced chest pain (e.g. angina), increased exercise capacity, increased cardiac ejection volume, decreased left ventricular end diastolic pressure, decreased pulmonary capillary wedge pressure, increased cardiac output, increased cardiac index, lowered pulmonary artery pressures, decreased left ventricular end systolic and diastolic dimensions, decreased left and right ventricular wall stress, decreased wall tension, increased quality of life, and decreased disease related morbidity or mortality. In one embodiment, modulation of one or more miRNAs in the heart cells of a subject suffering from myocardial ischemia can prevent the development of a myocardial infarct. In another embodiment, modulation of one or more miRNAs in the heart cells of a subject suffering from myocardial ischemia can limit the size of any subsequently occurring infarct by decreasing the loss of heart cells (e.g. decreasing apoptosis in the ischemic zone). In still another embodiment, cardiac function is stabilized in a subject suffering from myocardial ischemia following modulation of one or more miRNAs in the heart cells of the subject.

In certain embodiments, a subject in need of treatment or prevention of myocardial ischemia is a subject that is at a risk for a heart attack. For instance, in one embodiment, the subject has coronary artery disease. In some embodiments, the subject may exhibit one or more risk factors for coronary artery disease including, but not limited to, hypertension, hypercholesterolemia, smoking, hyperglycemia, diabetes mellitus, unstable angina, past experience of heart attacks, and familial history of heart disease.

The present invention also includes a method of treating or preventing ischemia-reperfusion injury in a subject in need thereof. As used herein, “ischemia-reperfusion injury” refers to tissue damage caused by restoration of blood flow following a period of ischemia. Restoration of blood flow after a period of ischemia can actually be more damaging than that resulting from the ischemia itself. Reintroduction of circulation to the ischemic tissue induces oxidative stress resulting in inflammation and oxidative damage through a greater production of damaging free radicals. Tissue necrosis can be greatly accelerated with reperfusion injury.

In one embodiment, the method comprises modulating the expression or activity of one or more miRNAs listed in Table 2 and the human counterparts thereof in the heart cells of the subject. In certain embodiments, the method comprises administering to the subject an inhibitor of one or more miRNAs listed in Table 2. In one embodiment, the inhibitor is an inhibitor of the expression or activity of one or more of a miR-15 family member (e.g. miR-15a, miR-15b, miR-16-1, miR-16-2, miR-195, miR-424 and miR-497) (SEQ ID NOs: 1-6) and miR-92a (SEQ ID NO: 16). In other embodiments, the method comprises administering to the subject an agonist of one or more miRNAs listed in Table 2. In one embodiment, the agonist is an agonist of one or more of miR-22 (SEQ ID NO: 36), miR-126 (SEQ ID NO: 20), and miR-29b (SEQ ID NO: 34). In another embodiment, the agonist or inhibitor of one or more miRNAs is administered to the ischemic tissue. In yet another embodiment, the agonist or inhibitor of one or more miRNAs is administered to the non-ischemic tissue bordering the ischemic tissue. Any of the agonists or inhibitors of miRNA function or expression as described herein is suitable for use in the methods of treating or preventing ischemia-reperfusion injury.

The present invention contemplates the use of agonists and inhibitors of identified miRNAs in the treatment and prevention of myocardial ischemia and ischemia-reperfusion injury in a subject. Treatment regimens would vary depending on the clinical situation, with earliest intervention being sought. However, long-term maintenance for at least some period of time after an ischemic event would appear to be appropriate in most circumstances. It also may be desirable to treat with modulators of miRNAs intermittently, or to vary which miRNAs are given, in order to maximize the protective effects.

In certain embodiments, it is envisioned to use a modulator of miRNA function or expression in combination with other therapeutic modalities. Thus, in addition to the miRNA therapies described above, one may also provide to the subject one or more “standard” pharmaceutical cardiac therapies. Examples of other therapies include, without limitation, beta-blockers, anti-hypertensives, cardiotonics, anti-thrombotics, vasodilators, hormone antagonists, iontropes, diuretics, endothelin receptor antagonists, calcium channel blockers, phosphodiesterase inhibitors, ACE inhibitors, angiotensin type 2 antagonists and cytokine blockers/inhibitors, and HDAC inhibitors.

In certain embodiments, administration of an agent that lowers the concentration of one of more blood lipids and/or lipoproteins, known herein as an “antihyperlipoproteinemic,” may be combined with a cardiovascular therapy according to the present invention (e.g. miRNA modulator), particularly in treatment of atherosclerosis and thickenings or blockages of vascular tissues. In certain embodiments, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof. Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate. Non-limiting examples of resins/bile acid sequesterants include cholestyramine (cholybar, questran), colestipol (colestid) and polidexide. Non-limiting examples of HMG CoA reductase inhibitors include lovastatin (mevacor), pravastatin (pravochol) or simvastatin (zocor). Non-limiting examples of nicotinic acid derivatives include nicotinate, acepimox, niceritrol, nicoclonate, nicomol and oxiniacic acid. Non-limiting examples of thyroid hormones and analogs thereof include etoroxate, thyropropic acid and thyroxine.

In certain embodiments, a miRNA modulator can be combined with an antiarrhythmic agent for the treatment of cardiovascular conditions. Non-limiting examples of antiarrhythmic agents include Class I antiarrhythmic agents (sodium channel blockers), Class II antiarrhythmic agents (beta-adrenergic blockers), Class III antiarrhythmic agents (repolarization prolonging drugs), Class IV antiarrhythmic agents (calcium channel blockers) and miscellaneous antiarrhythmic agents.

Sodium channel blockers include, but are not limited to, Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).

Exemplary beta blockers, otherwise known as a β-adrenergic blockers, β-adrenergic antagonists or Class II antiarrhythmic agents, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain embodiments, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

Examples of Class III antiarrhythmic agents include agents that prolong repolarization, such as amiodarone (cordarone) and sotalol (β-pace). Non-limiting examples of Class IV antiarrythmic agents, also known as calcium channel blockers, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (nifedipine-type) calcium antagonist.

Suitable examples of miscellaneous antiarrhythmic agents that can also be combined with a miRNA modulator of the invention include, but are not limited to, adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

In yet another embodiment of the invention, the miRNA modulator can be administered in combination with an antihypertensive agent. Non-limiting examples of antihypertensive agents include sympatholytic, alpha/beta blockers, alpha blockers, anti-angiotensin II agents, beta blockers, calcium channel blockers, vasodilators and miscellaneous antihypertensives.

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin. In certain embodiments, an antihypertensive agent is both an alpha and beta adrenergic antagonist. Non-limiting examples of an alpha/beta blocker comprise labetalol (normodyne, trandate).

Non-limiting examples of anti-angiotensin II agents include angiotensin converting enzyme inhibitors and angiotensin II receptor antagonists. Non-limiting examples of angiotensin converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotensin II receptor antagonist, an ANG receptor blocker or an ANG-II type-1 receptor blocker (ARBS), include angiocandesartan, eprosartan, irbesartan, losartan and valsartan.

In certain embodiments a cardiovasculator therapeutic agent may comprise a vasodilator (e.g., a cerebral vasodilator, a coronary vasodilator or a peripheral vasodilator) that can be co-administered with a miRNA modulator of the invention. In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimefylline, trapidil, tricromyl, trimetazidine, trolnitrate phosphate and visnadine.

In certain embodiments, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

A miRNA modulator of the invention can be combined with an inotropic agent. In some embodiments, the inotropic agent is a positive inotropic agent. Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular embodiments, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

In certain embodiments, a miRNA modulator of the invention is co-administered with endothelin for treatment of a cardiovascular disease. Endothelin (ET) is a 21-amino acid peptide that has potent physiologic and pathophysiologic effects that appear to be involved in the development of heart failure. The effects of ET are mediated through interaction with two classes of cell surface receptors. The type A receptor (ET-A) is associated with vasoconstriction and cell growth while the type B receptor (ET-B) is associated with endothelial-cell mediated vasodilation and with the release of other neurohormones, such as aldosterone. Pharmacologic agents that can inhibit either the production of ET or its ability to stimulate relevant cells are known in the art Inhibiting the production of ET involves the use of agents that block an enzyme termed endothelin-converting enzyme that is involved in the processing of the active peptide from its precursor. Inhibiting the ability of ET to stimulate cells involves the use of agents that block the interaction of ET with its receptors. Non-limiting examples of endothelin receptor antagonists (ERA) include Bosentan, Enrasentan, Ambrisentan, Darusentan, Tezosentan, Atrasentan, Avosentan, Clazosentan, Edonentan, sitaxsentan, TBC 3711, BQ 123, and BQ 788.

In certain embodiments, the secondary therapeutic agent that can be combined with the miRNA modulator may comprise a surgery of some type, which includes, for example, preventative, diagnostic or staging, curative and palliative surgery. Surgery, and in particular a curative surgery, may be used in conjunction with other therapies, such as the miRNA modulators of the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Combinations may be achieved by contacting cardiac cells with a single composition or a pharmacological formulation that includes one or more miRNA modulators and a second cardiac therapeutic agent, or by contacting the cell with two distinct compositions or formulations, at the same time, wherein one composition includes one or more miRNA modulators and the other includes the second cardiac therapeutic agent. Alternatively, administration of one or miRNA modulators may precede or follow administration of the other cardiac agent(s) by intervals ranging from minutes to weeks. In embodiments where the other cardiac agent and one or miRNA modulators are applied separately to the subject, one would generally ensure that a significant period of time did not expire between the time of each delivery, such that the cardiac agent and one or miRNA modulators would still be able to exert an advantageously combined effect on the cell. In such instances, it is contemplated that one would typically administer the two compositions within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a modulator of one or more miRNAs, or the other cardiac agent will be desired. In this regard, various combinations may be employed. By way of illustration, where the miRNA modulator is “A” and the other cardiac agent is “B,” the following permutations based on 3 and 4 total administrations are exemplary:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations are likewise contemplated.

In another embodiment, the present invention provides a method of preventing or reducing cardiomyocyte loss in response to hypoxia in a subject in need thereof. “Hypoxia” refers to a condition in which a tissue is not receiving an adequate supply of oxygen to satisfy the oxygen demand of the tissue. Prolonged hypoxia can lead to cell death. In one embodiment, the method comprises administering an inhibitor of miR-199a (e.g., SEQ ID NOs: 8-9), miR-320 (e.g., SEQ ID NO: 10), and/or an agonist of miR-210 (e.g., SEQ ID NO: 32) to the subject. The inhibitor of miR-199a or miR-320 function or expression can be any of the inhibitors disclosed herein. For instance, the inhibitor of miR-199a or miR-320 can be an antagomir or antisense oligonucleotide comprising a sequence that is at least partially complementary to the mature miR-199a or miR-320 sequence.

An agonist of miR-210 function or expression can be any of the agonists disclosed herein. For instance, in one embodiment, the agonist of miR-210 is a polynucleotide comprising a mature sequence of miR-210. In another embodiment, the agonist of miR-210 is the transcription factor, HIF1α. In certain embodiments, the agonist of miR-210 is expressed from an expression construct.

Pharmacological therapeutic agents and methods of administration, dosages, etc., are well known to those of skill in the art (see for example, the “Physicians Desk Reference”, Klaassen's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Where clinical applications are contemplated, pharmaceutical compositions comprising a modulator of one or miRNAs identified in Tables 1 and 2 will be prepared in a form appropriate for the intended application. Generally, this will entail preparing compositions that are essentially free of pyrogens, as well as other impurities that could be harmful to humans or animals. Colloidal dispersion systems, such as macromolecule complexes, nanocapsules, microspheres, beads, and lipid-based systems including oil-in-water emulsions, micelles, mixed micelles, and liposomes, may be used as delivery vehicles for the oligonucleotide inhibitors of miRNA function or miRNA agonists (e.g. constructs expressing particular miRNAs or polynucleotides encoding miRNAs). Commercially available fat emulsions that are suitable for delivering the nucleic acids of the invention to tissues, such as cardiac muscle tissue, include Intralipid®, Liposyn®, Liposyn® II, Liposyn® III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO 03/093449, which are herein incorporated by reference in their entireties.

One will generally desire to employ appropriate salts and buffers to render nucleic acids, agonists, inhibitors, and delivery vectors stable and allow for uptake by target cells. Buffers also will be employed when recombinant cells are introduced into a patient. Aqueous compositions of the present invention comprise an effective amount of the agent, dissolved or dispersed in a pharmaceutically acceptable carrier or aqueous medium. The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or nucleic acids of the compositions.

The active compositions of the present invention may include classic pharmaceutical preparations. Administration of these compositions according to the present invention may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Pharmaceutical compositions comprising miRNA inhibitors or agonists may also be administered by catheter systems or systems that isolate coronary circulation for delivering therapeutic agents to the heart. Various catheter systems for delivering therapeutic agents to the heart and coronary vasculature are known in the art. Some non-limiting examples of catheter-based delivery methods or coronary isolation methods suitable for use in the present invention are disclosed in U.S. Pat. No. 6,416,510; U.S. Pat. No. 6,716,196; U.S. Pat. No. 6,953,466, WO 2005/082440, WO 2006/089340, U.S. Patent Publication No. 2007/0203445, U.S. Patent Publication No. 2006/0148742, and U.S. Patent Publication No. 2007/0060907, which are all herein incorporated by reference in their entireties. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It is contemplated that any embodiment discussed herein can be implemented with respect to any method or composition of the invention, and vice versa. Furthermore, compositions of the invention can be used to achieve methods of the invention.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

### EXAMPLES

### Example 1

### Identification of miRNAs That are Regulated During Short Term Ischemia

Cardiac ischemia induces remodeling that can influence the function of the ventricle and the prognosis for survival. To determine whether miRNAs are involved in the different remodeling processes following an ischemic event, a miRNA microarray analysis was performed on tissue isolated from the infarcted zone at 6, 24 and 48 hours after an ischemic insult. Specifically, myocardial ischemia was induced in mice by occluding the left anterior descending artery, and miRNA expression profiles of tissue in the ischemic region at 6, 24, and 48 hours following induction were compared to the expression profile of myocardial tissue from sham operated animals. MiRNAs that were significantly regulated in the short term following the ischemic insult are listed in Table 1. The data are presented as the absolute value of miR expression in either sham operated animals or 6, 24 or 48 hours after myocardial ischemia. The expression profile at the different time points varies considerably for each specific miRNA, indicating a very dynamic and specific effect of miRNA contribution to cardiac remodeling in response to ischemia.

TABLE 1
Significantly regulated miRNAs in response to ischemia

| | | | 6 hours | 24 hours | 48 |
|---|---|---|---|---|---|
| | | Sham- | post- | post- | hours |
| No. Reporter | | operated | MI | MI | post-MI |
| Name | p-value | Mean | Mean | Mean | Mean |
| mmu-miR-1892 | 0.00E+00 | 173 | 1,421 | 42,346 | 6,642 |
| mmu-miR-574-5p | 0.00E+00 | 28 | 462 | 7,108 | 2,350 |
| mmu-miR-1187 | 0.00E+00 | 19 | 259 | 6,489 | 2,773 |
| mmu-miR-711 | 0.00E+00 | 6 | 9 | 5,501 | 456 |
| mmu-miR-1196 | 0.00E+00 | 113 | 241 | 3,040 | 2,188 |
| mmu-miR-705 | 5.55E−16 | 734 | 2,944 | 20,067 | 5,282 |
| mmu-miR- | 5.55E−16 | 13 | 111 | 545 | 204 |
| 1897-5p | | | | | |
| mmu-miR-21 | 6.66E−16 | 1,594 | 316 | 742 | 11,048 |
| mmu-miR-29a | 8.88E−16 | 3,478 | 1,370 | 108 | 390 |
| mmu-miR-483 | 1.55E−15 | 36 | 492 | 4,028 | 1,454 |
| mmu-miR-466i | 1.78E−15 | 11 | 96 | 760 | 407 |
| mmu-miR-574-3p | 1.78E−15 | 20 | 86 | 532 | 313 |
| mmu-miR-714 | 2.00E−15 | 26 | 211 | 957 | 118 |
| mmu-miR-1895 | 2.44E−15 | 112 | 727 | 2,513 | 1,021 |
| mmu-miR- | 2.89E−15 | 81 | 101 | 763 | 205 |
| 1894-3p | | | | | |
| mmu-miR- | 1.24E−14 | 31 | 391 | 1,409 | 687 |
| 466f-3p | | | | | |
| mmu-miR-30e | 1.32E−14 | 734 | 199 | 32 | 49 |
| mmu-miR-30c | 1.75E−14 | 3,802 | 2,887 | 374 | 640 |
| mmu-miR-30b | 7.23E−14 | 2,940 | 1,486 | 170 | 383 |
| mmu-miR-762 | 8.72E−14 | 668 | 5,020 | 36,127 | 10,932 |
| mmu-miR-467f | 1.91E−13 | 25 | 275 | 2,079 | 972 |
| mmu-miR- | 3.06E−13 | 3,036 | 2,482 | 507 | 1,866 |
| 125b-5p | | | | | |
| mmu-miR-27b | 5.30E−13 | 2,137 | 1,390 | 250 | 548 |
| mmu-miR-671-5p | 8.04E−13 | 49 | 472 | 486 | 85 |
| mmu-let-7e | 8.34E−13 | 1,359 | 542 | 113 | 201 |
| mmu-miR-689 | 1.46E−12 | 224 | 2,984 | 1,510 | 811 |
| mmu-let-7g | 2.16E−12 | 3,344 | 2,578 | 470 | 839 |
| mmu-miR-30a | 2.33E−12 | 1,722 | 801 | 174 | 235 |
| mmu-miR-126-3p | 7.99E−12 | 15,856 | 17,569 | 6,483 | 19,543 |
| mmu-miR- | 9.40E−12 | 740 | 360 | 56 | 339 |
| 125a-5p | | | | | |
| mmu-miR-1224 | 1.80E−11 | 565 | 1,274 | 2,673 | 782 |
| mmu-miR-290-5p | 1.95E−11 | 22 | 31 | 438 | 113 |
| mmu-miR-27a | 2.63E−11 | 1,437 | 594 | 160 | 300 |
| mmu-miR-690 | 3.25E−11 | 282 | 428 | 936 | 1,361 |
| mmu-miR-22 | 8.60E−11 | 554 | 394 | 110 | 138 |
| mmu-miR-133b | 9.55E−11 | 2,406 | 2,883 | 428 | 1,001 |
| mmu-miR-92a | 1.57E−10 | 290 | 166 | 242 | 531 |
| mmu-miR- | 1.70E−10 | 740 | 555 | 180 | 530 |
| 199a-3p | | | | | |
| mmu-miR-451 | 5.79E−10 | 2,026 | 1,104 | 4,410 | 7,011 |
| mmu-miR-26b | 1.01E−09 | 2,435 | 1,944 | 412 | 818 |
| mmu-miR-1 | 1.67E−09 | 47,997 | 56,431 | 17,333 | 42,200 |
| mmu-miR-709 | 1.81E−09 | 10,451 | 14,051 | 25,010 | 23,279 |
| mmu-let-7a | 1.89E−09 | 9,285 | 8,734 | 2,867 | 4,867 |
| mmu-miR-133a | 3.33E−09 | 2,523 | 3,336 | 549 | 1,164 |
| mmu-miR-499 | 4.58E−09 | 1,309 | 420 | 6 | 7 |
| mmu-let-7d | 4.95E−09 | 6,334 | 6,018 | 2,034 | 3,027 |
| mmu-miR-30d | 5.62E−09 | 627 | 485 | 148 | 343 |
| mmu-let-7f | 5.74E−09 | 8,196 | 8,693 | 2,567 | 4,218 |
| mmu-miR-195 | 6.08E−09 | 1,141 | 861 | 267 | 482 |
| mmu-miR-15b | 6.56E−09 | 381 | 226 | 284 | 797 |
| mmu-miR-150 | 8.25E−09 | 426 | 434 | 54 | 225 |
| mmu-miR-151-5p | 1.09E−08 | 508 | 464 | 154 | 318 |
| mmu-let-7b | 1.77E−08 | 5,171 | 6,240 | 2,107 | 3,528 |
| mmu-miR-25 | 3.60E−08 | 335 | 222 | 297 | 625 |
| mmu-miR-26a | 4.31E−08 | 11,427 | 12,159 | 5,297 | 11,307 |
| mmu-miR-214 | 7.41E−08 | 335 | 356 | 786 | 1,027 |
| mmu-let-7c | 1.33E−07 | 8,677 | 9,349 | 3,156 | 5,642 |
| mmu-miR-805 | 1.82E−07 | 2,455 | 2,287 | 1,472 | 754 |
| mmu-miR-23b | 2.68E−07 | 7,231 | 7,438 | 4,570 | 9,090 |
| mmu-miR-23a | 2.82E−07 | 6,448 | 6,714 | 4,965 | 10,303 |
| mmu-let-7i | 3.90E−07 | 2,991 | 3,601 | 1,535 | 3,090 |
| mmu-miR-16 | 2.09E−06 | 2,552 | 2,033 | 1,126 | 1,893 |
| mmu-miR-486 | 4.16E−06 | 643 | 454 | 280 | 662 |
| mmu-miR-24 | 5.58E−06 | 2,347 | 2,891 | 1,646 | 2,164 |
| mmu-miR-378 | 1.01E−05 | 1,526 | 1,683 | 1,432 | 934 |
| mmu-miR-143 | 1.44E−05 | 1,502 | 1,311 | 698 | 768 |
| mmu-miR-191 | 3.16E−05 | 582 | 505 | 295 | 531 |
| mmu-miR-29c | 1.79E−04 | 934 | 122 | 7 | 11 |

Interestingly, the significantly regulated miRNAs include several miRNAs that were also found to be regulated in our previous stress studies. For example, miR-21 and miR-574 were also highly induced in tissue isolated from the border zone of the infarct both 3 and 14 days post-MI (van Rooij et al. (2008) Proc. Natl. Acad. Sci., Vol. 105: 13027-13032). In the short term following an ischemic insult, miR-574 appeared to peak at 24 hrs, while expression of miR-21 decreased in the first 24 hrs, but was strongly expressed 48 hours after the ischemic event. In addition, these data show that the expression of miR-29, which has been reported to regulate collagen deposition and fibrosis (van Rooij et al. (2006) Proc. Natl. Acad. Sci., Vol. 103:18255-18260; van Rooij et al. (2008) Proc. Natl. Acad. Sci., Vol. 105: 13027-13032), was significantly reduced in the first 24 hours after the ischemic event. Also multiple members of the miR-30 family showed a strong decrease in expression in response to ischemia.

MiR-126, a endothelial specific miRNA (Wang et al., (2008) Dev. Cell, Vol. 15:261-271), was strongly downregulated in the first 24 hours, but appeared to increase in expression 48 hrs after MI. This pattern of expression may be explained by the reported role for miR-126 in neoangiogenesis (Wang et al., (2008) Dev. Cell, Vol. 15:261-271). MiR-92a, a miR previously implicated in angiogenesis (Bonauer et al. (2009) Science, Vol. 324 (5935):1710-1713), exhibited a similar expression profile in response to an ischemic event as miR-126, suggesting that miR-92a may also be influencing neoangiogenesis.

The skeletal muscle specific miRNAs, miR-1, miR-133, and miR-499, were differentially regulated following ischemia. For instance, the expression of miR-1 and miR-133 was decreased after 24 hours, but rebounded 48 hours after the ischemic event. In contrast, the expression of miR-499 was suppressed for the first 48 hours after ischemia.

The expression of miR-15 family members (miR-15a/b, miR-16, and miR-195), which have been implicated in the regulation of cell survival and proliferation (see, e.g., WO 2009/062169), showed an initial decrease following myocardial ischemia. Inhibition of these miRNAs would be beneficial to enhance cell survival in the ischemic region in response to MI thus limiting the size of any potential resulting infarct. Another familiar stress responsive miR, miR-214, was induced in response to ischemia and may play a role in muscle cell proliferation and fate determination (Watanabe T et al., (2008) Dev. Dyn., Vol. 237:3738-3748; Flynt A S et al. (2007) Nat Genet., Vol. 39:259-63). MiR-143, which is a vascular smooth muscle cell specific miRNA that plays a role in smooth muscle proliferation (see pending application PCT/US09/34887), was significantly downregulated in the ischemic region at both 24 and 48 hours following an ischemic event. A decrease in miR-143 increases the proliferation of smooth muscle cells and is detrimental to the heart. Thus, increasing the expression of miR-143 would act to control smooth muscle cell proliferation and promote recovery of function to the ischemic tissue.

### Example 2

### MiR-199 Regulates HIF1 Alpha and miR-210 in Ischemic Tissue

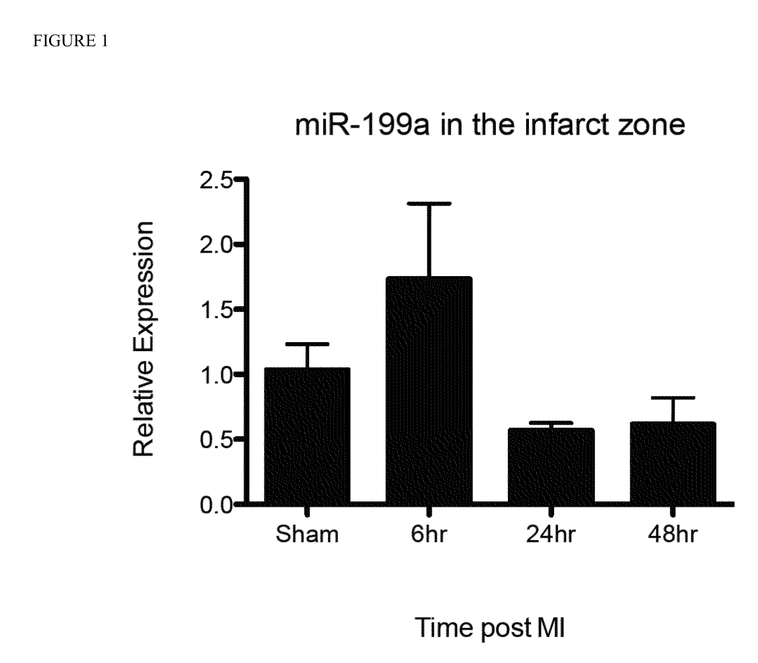

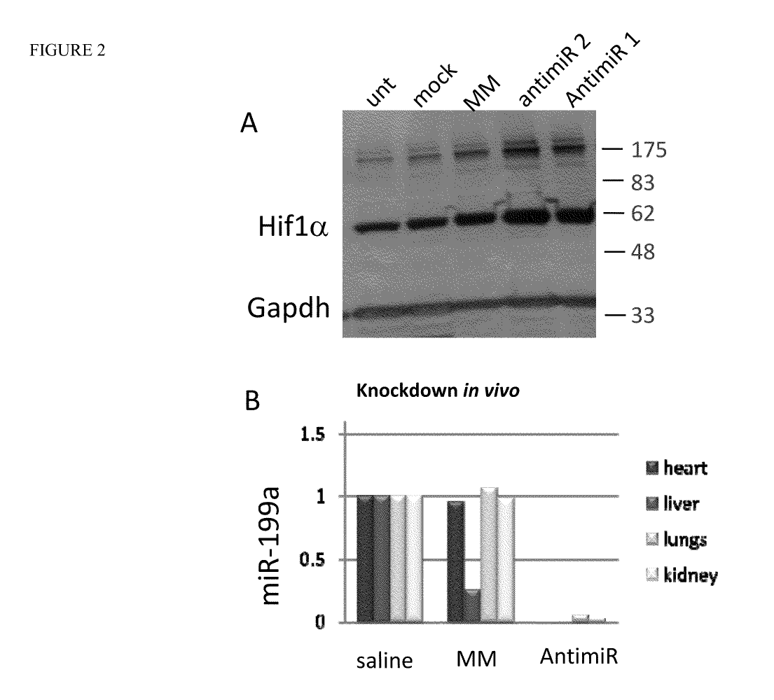

Expression of miR-199a in the ischemic tissue was reduced in the first 24 hours following ischemia, but began to show recovery 48 hours after the ischemic event. Realtime PCR analysis of miR-199 confirms that miR-199a expression is significantly reduced in the ischemic tissue 24 hours after an ischemic insult (FIG. 1). Interestingly, we previously identified miR-199 as a miRNA that was regulated in the border zone of a myocardial infarct. In response to MI, miR-199 was upregulated both 3 and 14 days post-MI (van Rooij et al. (2006) Proc. Natl. Acad. Sci., Vol. 103:18255-18260; van Rooij et al. (2008) Proc. Natl. Acad. Sci., Vol. 105: 13027-13032). To further elucidate the role of miR-199 following ischemia, we identified the transcription factor, hypoxia-inducible factor 1 alpha (HIF1α) as a target of miR-199. To confirm whether HIF1α was a functional target of miR-199, cardiomyocytes were treated with oligonucleotides that had a complementary sequence to that of miR-199a (antimiRs) or controls that had a mismatched sequence (MM). HIF1α expression, as assessed by Northern blot, was increased in cardiomyoctyes that were treated with the antimiRs for miR-199a, while no change in expression was observed in cardiomyocytes treated with the mismatched control (FIG. 2A). These results suggest that a decrease in expression of miR-199a would cause an increase in HIF1α levels, which in turn would activate expression of genes that control the hypoxic response. Such an increase in HIF1α expression would be beneficial after an ischemic insult.

To demonstrate that antimiRs directed to miR-199a could efficiently knockdown miR-199 in vivo, mice were injected intravenously with an antimiR against miR-199a. Realtime PCR analysis of heart, lung, liver, and kidney tissue showed that injection of antimiR-199a produced an almost complete knockdown of miR-199 in all tissues measured as compared to saline-injected animals (FIG. 2B). Injection of a mismatched control produced some knockdown of miR-199 in liver tissue, but not in the other tissues measured.

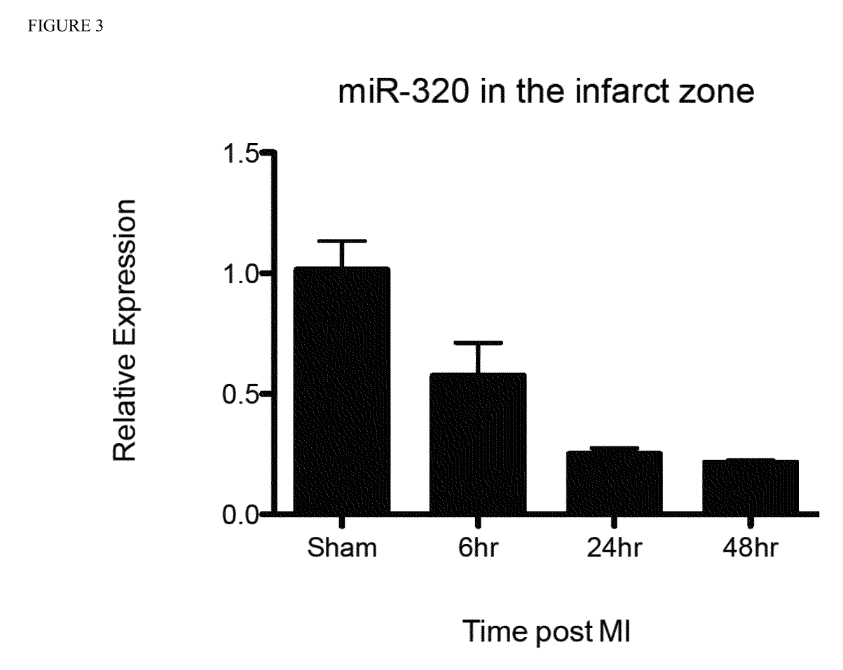

MiR-320 has also been reported to be downregulated following hypoxia. We examined the expression of miR-320 by realtime PCR analysis in ischemic tissue to determine whether this miRNA was also regulated in cardiac tissue in response to ischemia. As shown in FIG. 3, expression of miR-320 is significantly reduced as early as six hours following induction of myocardial ischemia. MiR-320 targets heat shock protein 20 (HSP20), which has been implicated in enhancement of myocardial function (Fan et al. (2007) Circulation, Vol. 116: II-189). Thus, the decrease in miR-320 expression would result in an upregulation of HSP20 and a corresponding enhancement of myocardial function. Further downregulation of miR-320 expression would be therapeutic following ischemia.

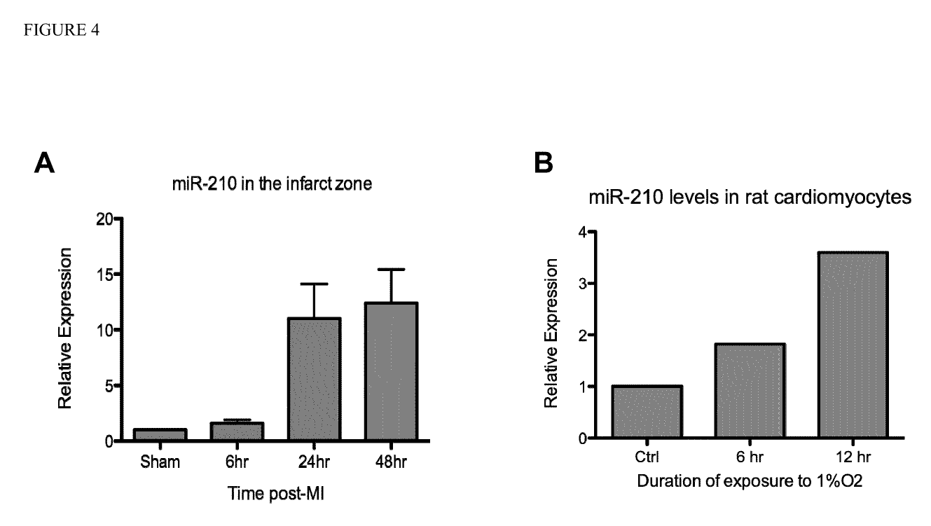

Recently, miR-210 has been implicated in the endothelial cell response to hypoxia (Fasanaro et al. (2008) J. Biol. Chem., Vol. 283:15878-15883) and is thought to be downstream of HIF1α signaling. To determine whether miR-210 was regulated in cardiac tissue in response to ischemia, we examined the expression of miR-210 by realtime PCR analysis in ischemic tissue following induction of a myocardial ischemia. As shown in FIG. 4A, miR-210 expression is strongly induced 24 hours after the ischemic event. We also observed a similar induction of miR-210 in rat neonatal cardiomyocytes exposed to hypoxic conditions in vitro (FIG. 4B). MiR-210 has been reported to decrease pro-apoptotic signaling and thus functions as an anti-apoptotic factor (Kulshreshtha et al. (2007) Mol. Cell Biol., Vol. 27:1859-1867). Therefore, it is likely that induction of miR-210 following ischemia confers protection of heart cells by preventing apoptosis and loss of cardiomyocytes.

### Example 3

### Identification of miRNAs Regulated Following Ischemia Reperfusion Injury

To further examine the role of miRNAs in cardiac remodeling following ischemic injury, a miRNA microarray was performed on cardiac tissue following ischemia reperfusion. During myocardial ischemia the blood supply to the mitochondria in the infarcted region is inadequate to support oxidative phosphorylation. Ischemia is often followed by reperfusion allowing the re-admission of oxygen and metabolic substrates which replaces the ischemic metabolites. The process of reperfusion induces biochemical, structural and functional changes in the myocardium and may determine cell survival and cell death. Regulation of this process may decrease the deleterious effects of ischemia and/or reperfusion and thereby enhance the clinical outcome of myocardial infarction.

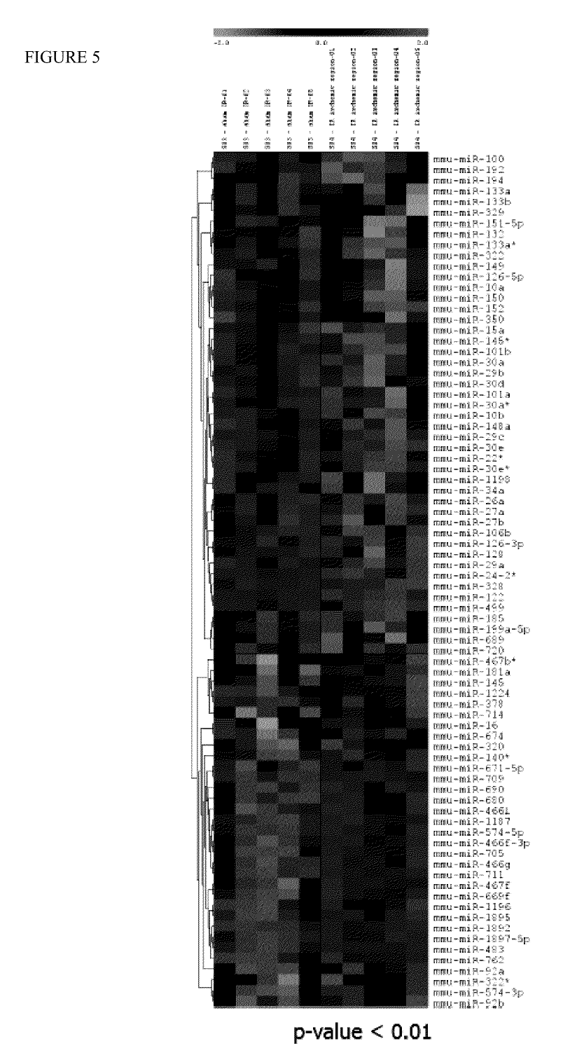

Specifically, male C57B16 mice were subject to 45 minutes of myocardial ischemia. The tissue was then allowed to be reperfused for 24 hours. Tissue was collected from the ischemic-reperfused region and subject to miRNA microarray analysis. Several miRNAs were found to be regulated following reperfusion of the ischemic tissue (Table 2 and FIG. 5).

TABLE 2
Significantly regulated miRNAs in response to ischemia reperfusion

| | | Sham- | Ischemia/ | Log2 |
|---|---|---|---|---|
| | | operated | reperfusion | (ischemic/ |
| No. Reporter Name | p-value | Mean | Mean | sham) |
| mmu-miR-1892 | 2.49E−08 | 292 | 1,267 | 2.12 |
| mmu-miR-709 | 8.05E−08 | 15,600 | 28,596 | 0.87 |
| mmu-miR-499 | 1.86E−07 | 3,035 | 918 | −1.72 |
| mmu-miR-126-3p | 1.59E−06 | 39,722 | 27,548 | −0.53 |
| mmu-miR-762 | 1.79E−06 | 1,275 | 2,940 | 1.21 |
| mmu-miR-30e | 2.41E−06 | 1,175 | 485 | −1.28 |
| mmu-miR-29a | 2.83E−06 | 8,786 | 4,676 | −0.91 |
| mmu-miR-690 | 4.38E−06 | 350 | 689 | 0.98 |
| mmu-miR-26a | 7.04E−06 | 32,537 | 22,791 | −0.51 |
| mmu-miR-29c | 7.60E−06 | 2,073 | 597 | −1.80 |
| mmu-miR-27a | 2.62E−05 | 3,168 | 2,168 | −0.55 |
| mmu-miR-30d | 5.00E−05 | 1,885 | 1,336 | −0.50 |
| mmu-miR-30a | 5.64E−05 | 2,864 | 1,834 | −0.64 |
| mmu-miR-705 | 6.42E−05 | 689 | 2,265 | 1.72 |
| mmu-miR-27b | 1.48E−04 | 4,049 | 3,303 | −0.29 |
| mmu-miR-1224 | 1.64E−04 | 389 | 715 | 0.88 |
| mmu-miR-92a | 5.28E−04 | 672 | 832 | 0.31 |
| mmu-miR-150 | 6.09E−04 | 1,123 | 809 | −0.47 |
| mmu-miR-133a | 6.15E−04 | 10,020 | 7,546 | −0.41 |
| mmu-miR-689 | 1.00E−03 | 473 | 326 | −0.54 |
| mmu-miR-320 | 1.01E−03 | 296 | 439 | 0.57 |
| mmu-miR-145 | 1.55E−03 | 1,579 | 2,223 | 0.49 |
| mmu-miR-133b | 1.65E−03 | 9,107 | 7,226 | −0.33 |
| mmu-miR-378 | 2.79E−03 | 1,126 | 1,344 | 0.25 |
| mmu-miR-151-5p | 5.16E−03 | 805 | 689 | −0.22 |
| mmu-miR-16 | 8.28E−03 | 4,093 | 5,039 | 0.30 |
| mmu-miR-122 | 1.39E−08 | 296 | 17 | −4.13 |
| mmu-miR-328 | 5.65E−08 | 94 | 44 | −1.12 |
| mmu-miR-1187 | 1.69E−07 | 53 | 312 | 2.55 |
| mmu-miR-1897-5p | 2.48E−07 | 16 | 84 | 2.37 |
| mmu-miR-574-5p | 5.92E−07 | 58 | 370 | 2.68 |
| mmu-miR-24-2* | 1.96E−06 | 122 | 58 | −1.06 |
| mmu-miR-671-5p | 2.38E−06 | 28 | 69 | 1.27 |
| mmu-miR-466g | 3.28E−06 | 8 | 49 | 2.64 |
| mmu-miR-1895 | 4.99E−06 | 92 | 189 | 1.04 |
| mmu-miR-10b | 6.08E−06 | 120 | 43 | −1.50 |
| mmu-miR-29b | 7.28E−06 | 82 | 19 | −2.11 |
| mmu-miR-22* | 7.79E−06 | 95 | 43 | −1.16 |
| mmu-miR-711 | 8.78E−06 | 18 | 195 | 3.48 |
| mmu-miR-466f-3p | 9.51E−06 | 30 | 160 | 2.41 |
| mmu-miR-30e* | 1.02E−05 | 263 | 77 | −1.78 |
| mmu-miR-680 | 1.02E−05 | 15 | 60 | 2.00 |
| mmu-miR-466i | 1.10E−05 | 18 | 100 | 2.49 |
| mmu-miR-106b | 1.21E−05 | 91 | 73 | −0.32 |
| mmu-miR-148a | 1.90E−05 | 160 | 69 | −1.22 |
| mmu-miR-483 | 1.94E−05 | 43 | 307 | 2.84 |
| mmu-miR-1196 | 2.32E−05 | 219 | 345 | 0.65 |
| mmu-miR-140* | 2.74E−05 | 46 | 68 | 0.56 |
| mmu-miR-199a-5p | 3.58E−05 | 35 | 23 | −0.60 |
| mmu-miR-15a | 3.91E−05 | 225 | 127 | −0.83 |
| mmu-miR-720 | 6.86E−05 | 110 | 82 | −0.42 |
| mmu-miR-101b | 6.90E−05 | 45 | 19 | −1.22 |
| mmu-miR-30a* | 7.46E−05 | 226 | 106 | −1.10 |
| mmu-miR-145* | 8.65E−05 | 56 | 14 | −1.99 |
| mmu-miR-152 | 9.80E−05 | 241 | 165 | −0.55 |
| mmu-miR-669f | 1.42E−04 | 11 | 67 | 2.55 |
| mmu-miR-10a | 1.55E−04 | 52 | 21 | −1.33 |
| mmu-miR-101a | 1.60E−04 | 83 | 21 | −1.96 |
| mmu-miR-185 | 1.61E−04 | 315 | 263 | −0.26 |
| mmu-miR-34a | 1.62E−04 | 67 | 44 | −0.61 |
| mmu-miR-128 | 1.67E−04 | 278 | 192 | −0.53 |
| mmu-miR-1198 | 3.50E−04 | 43 | 30 | −0.53 |
| mmu-miR-467f | 3.69E−04 | 41 | 203 | 2.29 |
| mmu-miR-350 | 6.69E−04 | 35 | 11 | −1.60 |
| mmu-miR-192 | 6.87E−04 | 81 | 42 | −0.95 |
| mmu-miR-149 | 7.07E−04 | 56 | 36 | −0.64 |
| mmu-miR-126-5p | 7.33E−04 | 70 | 28 | −1.33 |
| mmu-miR-181a | 1.13E−03 | 166 | 211 | 0.35 |
| mmu-miR-322 | 1.16E−03 | 153 | 75 | −1.02 |
| mmu-miR-674 | 2.61E−03 | 68 | 79 | 0.21 |
| mmu-miR-133a* | 2.76E−03 | 69 | 51 | −0.44 |
| mmu-miR-100 | 3.39E−03 | 199 | 128 | −0.64 |
| mmu-miR-714 | 3.41E−03 | 36 | 53 | 0.56 |
| mmu-miR-132 | 4.20E−03 | 142 | 114 | −0.32 |
| mmu-miR-467b* | 4.96E−03 | 16 | 37 | 1.20 |
| mmu-miR-329 | 6.39E−03 | 82 | 49 | −0.76 |
| mmu-miR-194 | 6.89E−03 | 65 | 46 | −0.50 |
| mmu-miR-92b | 7.59E−03 | 233 | 282 | 0.27 |
| mmu-miR-574-3p | 8.44E−03 | 57 | 98 | 0.78 |
| mmu-miR-322* | 8.58E−03 | 30 | 35 | 0.23 |

These data indicate that miRNAs are regulated and actively involved in the process of cardiac remodeling in response to reperfusion following ischemia. Several of these miRNAs were also acutely regulated following an ischemic event (Example 1). Thus, there is a collection of miRNAs that are involved in the response of the heart to ischemia and subsequent reperfusion. Manipulating functionality of these specific miRNAs to control cardiac remodeling in response to ischemia can act to limit infarct size and maintain cardiac contractility, thereby providing a novel promising therapeutic approach to the treatment of myocardial infarction.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

### Sequence Listing

| | | SEQ |
|---|---|---|
| | | ID |
| Human miRNA | mature miRNA sequence | NO: |
| hsa-miR-15a | 5′-UAGCAGCACAUAAUGGUUUGUG-3′ | 1 |
| hsa-miR-15b | 5′-UAGCAGCACAUCAUGGUUUACA-3′ | 2 |
| hsa-miR-16-1/ | 5′-UAGCAGCACGUAAAUAUUGGCG-3′ | 3 |
| hsa-miR-16-2 | | |
| hsa-miR-195 | 5′-UAGCAGCACAGAAAUAUUGGC-3′ | 4 |
| hsa-miR-424 | 5′-CAGCAGCAAUUCAUGUUUUGAA-3′ | 5 |
| hsa-miR-497 | 5′-CAGCAGCACACUGUGGUUUGU-3′ | 6 |
| hsa-miR-21 | 5′-UAGCUUAUCAGACUGAUGUUGA-3′ | 7 |
| hsa-miR-199a-5p | 5′-CCCAGUGUUCAGACUACCUGUUC-3′ | 8 |
| hsa-miR-199a-3p | 5′-ACAGUAGUCUGCACAUUGGUUA-3′ | 9 |
| hsa-miR-320 | 5′-AAAAGCUGGGUUGAGAGGGCGA-3′ | 10 |
| hsa-miR-214 | 5′-ACAGCAGGCACAGACAGGCAGU-3′ | 11 |
| hsa-miR-10a | 5′-UACCCUGUAGAUCCGAAUUUGUG-3′ | 12 |
| hsa-miR-10b | 5′-UACCCUGUAGAACCGAAUUUGUG-3′ | 13 |
| hsa-miR-574-5p | 5′-UGAGUGUGUGUGUGUGAGUGUGU-3′ | 14 |
| hsa-miR-574-3p | 5′-CACGCUCAUGCACACACCCACA-3′ | 15 |
| hsa-miR-92a | 5′-UAUUGCACUUGUCCCGGCCUGU-3′ | 16 |
| hsa-miR-499-5p | 5′-UUAAGACUUGCAGUGAUGUUU-3′ | 17 |
| hsa-miR-499-3p | 5′-AACAUCACAGCAAGUCUGUGCU-3′ | 18 |
| hsa-miR-101 | 5′-UACAGUACUGUGAUAACUGAA-3′ | 19 |
| hsa-miR-126 | 5′-UCGUACCGUGAGUAAUAAUGCG-3′ | 20 |
| hsa-miR-30a | 5′-UGUAAACAUCCUCGACUGGAAG-3′ | 21 |
| hsa-miR-30b | 5′-UGUAAACAUCCUACACUCAGCU-3′ | 22 |
| hsa-miR-30c | 5′-UGUAAACAUCCUACACUCUCAGC-3′ | 23 |
| hsa-miR-30d | 5′-UGUAAACAUCCCCGACUGGAAG-3′ | 24 |
| hsa-miR-30e | 5′-UGUAAACAUCCUUGACUGGAAG-3′ | 25 |
| hsa-miR-143 | 5′-UGAGAUGAAGCACUGUAGCUC-3′ | 26 |
| hsa-miR-185 | 5′-UGGAGAGAAAGGCAGUUCCUGA-3′ | 27 |
| hsa-miR-34a | 5′-UGGCAGUGUCUUAGCUGGUUGU-3′ | 28 |
| hsa-miR-1 | 5′-UGGAAUGUAAAGAAGUAUGUAU-3′ | 29 |
| hsa-miR-133a | 5′-UUUGGUCCCCUUCAACCAGCUG-3′ | 30 |
| hsa-miR-133b | 5′-UUUGGUCCCCUUCAACCAGCUA-3′ | 31 |
| hsa-miR-210 | 5′-CUGUGCGUGUGACAGCGGCUGA-3′ | 32 |
| hsa-miR-29a | 5′-UAGCACCAUCUGAAAUCGGUUA-3′ | 33 |
| hsa-miR-29b | 5′-UAGCACCAUUUGAAAUCAGUGUU-3′ | 34 |
| hsa-miR-29c | 5′-UAGCACCAUUUGAAAUCGGUUA-3′ | 35 |
| hsa-miR-22 | 5′-AAGCUGCCAGUUGAAGAACUGU-3′ | 36 |
| hsa-miR-26a | 5′-UUCAAGUAAUCCAGGAUAGGCU-3′ | 37 |
| hsa-let-7b | 5′-UGAGGUAGUAGGUUGUGUGGUU-3′ | 38 |
| hsa-miR-125b | 5′-UCCCUGAGACCCUAACUUGUGA-3′ | 39 |
| hsa-miR-145 | 5′-GUCCAGUUUUCCCAGGAAUCCCU-3′ | 40 |

1. A method of treating or preventing myocardial ischemia in a subject in need thereof comprising modulating the expression or activity of one or more miRNAs listed in Tables 1 and 2 in the heart cells of the subject.

2. The method of claim 1, wherein the one or more miRNAs are selected from the group consisting of a miR-15 family member, miR-21, miR-26a, let-7b, miR-199a, miR-214, miR-10a, miR-10b, miR-574, miR-320, miR-92a, miR-499, miR-101a, miR-101b, miR-125b, miR-126, a miR-30 family member, miR-143, miR-145, miR-185, miR-34a, miR-1, miR-133, miR-210, and miR-29a-c.

3. The method of claim 2, wherein modulating comprises administering to the subject an inhibitor of one or more miRNAs selected from the group consisting of a miR-15 family member, miR-92a, miR-320, miR-21, miR-199a, miR-499, and a miR-30 family member.

4. The method of claim 3, wherein the inhibitor of one or more miRNAs is an antisense oligonucleotide or an antagomir.

5. The method of claim 4, wherein the antisense oligonucleotide comprises a sequence that is at least partially complementary to a mature sequence of said one or more miRNAs.

6. The method of claim 4, wherein the antisense oligonucleotide comprises at least one sugar and/or backbone modification.

7. The method of claim 4, wherein the antisense oligonucleotide is about 8 to about 18 nucleotides in length.

8. The method of claim 2, wherein modulating comprises administering to the subject an agonist of one or more miRNAs selected from the group consisting of miR-126, miR-143, miR-210, and miR-29a-c.

9. The method of claim 8, wherein the agonist of one or more miRNAs is a polynucleotide comprising a mature sequence of the one or more miRNAs.

10. The method of claim 9, wherein the agonist is expressed from an expression construct.

11. The method of claim 3, wherein the inhibitor is administered to the subject by intravenous administration, subcutaneous administration, or direct injection into cardiac tissue.

12. The method of claim 3, wherein the inhibitor is administered to the subject by oral, transdermal, sustained release, controlled release, delayed release, suppository, catheter or sublingual administration.

13. The method of claim 1, wherein the subject has coronary artery disease.

14. The method of claim 1, wherein cardiomyocyte loss is reduced or prevented in the subject following modulation of the expression or activity of one or more of the miRNAs.

15. The method of claim 1 further comprising administering a second cardiac therapeutic agent.

16. The method of claim 12, wherein the second cardiac therapeutic agent is selected from the group consisting of an antianginal agent, beta blocker, an ionotrope, a diuretic, ACE inhibitors, angiotensin type 2 antagonists, an endothelin receptor antagonist, an HDAC inhibitor, and a calcium channel blocker.

17. The method of claim 1, wherein the subject is human.

18. A method of preventing or reducing cardiomyocyte loss in response to hypoxia in a subject in need thereof comprising administering an inhibitor of miR-199a, miR-320, and/or an agonist of miR-210 to the subject.

19. The method of claim 18, wherein the inhibitor of miR-199a or miR-320 is an antisense oligonucleotide or an antagomir.

20. The method of claim 18, wherein the agonist of miR-210 is a polynucleotide comprising a mature sequence of miR-210.

21. The method of claim 18, wherein the agonist is HIF1α.

22. The method of claim 18, wherein the agonist is expressed from an expression construct.

23. The method of claim 8, wherein the agonist is administered to the subject by intravenous administration, subcutaneous administration, or direct injection into cardiac tissue.

24. The method of claim 8, wherein the agonist is administered to the subject by oral, transdermal, sustained release, controlled release, delayed release, suppository, catheter or sublingual administration.


SEQUENCE LISTING (40 sequences)

SEQ ID NO: 1 - RNA, 22 nt, Homo sapiens
uagcagcacauaaugguuugug

SEQ ID NO: 2 - RNA, 22 nt, Homo sapiens
uagcagcacaucaugguuuaca

SEQ ID NO: 3 - RNA, 22 nt, Homo sapiens
uagcagcacguaaauauuggcg

SEQ ID NO: 4 - RNA, 21 nt, Homo sapiens
uagcagcacagaaauauuggc

SEQ ID NO: 5 - RNA, 22 nt, Homo sapiens
cagcagcaauucauguuuugaa

SEQ ID NO: 6 - RNA, 21 nt, Homo sapiens
cagcagcacacugugguuugu

SEQ ID NO: 7 - RNA, 22 nt, Homo sapiens
uagcuuaucagacugauguuga

SEQ ID NO: 8 - RNA, 23 nt, Homo sapiens
cccaguguucagacuaccuguuc

SEQ ID NO: 9 - RNA, 22 nt, Homo sapiens
acaguagucugcacauugguua

SEQ ID NO: 10 - RNA, 22 nt, Homo sapiens
aaaagcuggguugagagggcga

SEQ ID NO: 11 - RNA, 22 nt, Homo sapiens
acagcaggcacagacaggcagu

SEQ ID NO: 12 - RNA, 23 nt, Homo sapiens
uacccuguagauccgaauuugug

SEQ ID NO: 13 - RNA, 23 nt, Homo sapiens
uacccuguagaaccgaauuugug

SEQ ID NO: 14 - RNA, 23 nt, Homo sapiens
ugagugugugugugugagugugu

SEQ ID NO: 15 - RNA, 22 nt, Homo sapiens
cacgcucaugcacacacccaca

SEQ ID NO: 16 - RNA, 22 nt, Homo sapiens
uauugcacuugucccggccugu

SEQ ID NO: 17 - RNA, 21 nt, Homo sapiens
uuaagacuugcagugauguuu

SEQ ID NO: 18 - RNA, 22 nt, Homo sapiens
aacaucacagcaagucugugcu

SEQ ID NO: 19 - RNA, 21 nt, Homo sapiens
uacaguacugugauaacugaa

SEQ ID NO: 20 - RNA, 22 nt, Homo sapiens
ucguaccgugaguaauaaugcg

SEQ ID NO: 21 - RNA, 22 nt, Homo sapiens
uguaaacauccucgacuggaag

SEQ ID NO: 22 - RNA, 22 nt, Homo sapiens
uguaaacauccuacacucagcu

SEQ ID NO: 23 - RNA, 23 nt, Homo sapiens
uguaaacauccuacacucucagc

SEQ ID NO: 24 - RNA, 22 nt, Homo sapiens
uguaaacauccccgacuggaag

SEQ ID NO: 25 - RNA, 22 nt, Homo sapiens
uguaaacauccuugacuggaag

SEQ ID NO: 26 - RNA, 21 nt, Homo sapiens
ugagaugaagcacuguagcuc

SEQ ID NO: 27 - RNA, 22 nt, Homo sapiens
uggagagaaaggcaguuccuga

SEQ ID NO: 28 - RNA, 22 nt, Homo sapiens
uggcagugucuuagcugguugu

SEQ ID NO: 29 - RNA, 22 nt, Homo sapiens
uggaauguaaagaaguauguau

SEQ ID NO: 30 - RNA, 22 nt, Homo sapiens
uuugguccccuucaaccagcug

SEQ ID NO: 31 - RNA, 22 nt, Homo sapiens
uuugguccccuucaaccagcua

SEQ ID NO: 32 - RNA, 22 nt, Homo sapiens
cugugcgugugacagcggcuga

SEQ ID NO: 33 - RNA, 22 nt, Homo sapiens
uagcaccaucugaaaucgguua

SEQ ID NO: 34 - RNA, 23 nt, Homo sapiens
uagcaccauuugaaaucaguguu

SEQ ID NO: 35 - RNA, 22 nt, Homo sapiens
uagcaccauuugaaaucgguua

SEQ ID NO: 36 - RNA, 22 nt, Homo sapiens
aagcugccaguugaagaacugu

SEQ ID NO: 37 - RNA, 22 nt, Homo sapiens
uucaaguaauccaggauaggcu

SEQ ID NO: 38 - RNA, 22 nt, Homo sapiens
ugagguaguagguugugugguu

SEQ ID NO: 39 - RNA, 22 nt, Homo sapiens
ucccugagacccuaacuuguga

SEQ ID NO: 40 - RNA, 23 nt, Homo sapiens
guccaguuuucccaggaaucccu